(19) United States
(12) Patent Application Publication
(10) Pub. No.: US 2026/0269071 A1
(43) Pub. Date: Sep. 10, 2026

(54) PATIENT-SPECIFIC TOTAL HIP ARTHROPLASTY PERIPROSTHETIC FRACTURE RISK CALCULATOR

(71) Applicant: Mayo Foundation for Medical Education and Research, Rochester, MN (US)

(72) Inventors: Cody C. Wyles, Rochester, MN (US); Bardia Khosravi, Rochester, MN (US); Pouria Rouzrokh, New Haven, CT (US)

(21) Appl. No.: 19/166,576

(22) PCT Filed: Mar. 20, 2024
(86) PCT No.: PCT/US2024/020689

§ 371 Date: Sep. 18, 2025

Related U.S. Application Data
(60) Provisional application No. 63/453,366, filed on Mar. 20, 2023.

(51) Int. Cl. G16H 50/30

(52) U.S. Cl. CPC G16H 50/30

(57) ABSTRACT

Provided herein is a computer-implemented method including obtaining values for non-modifiable risk factors, each defining an immutable patient characteristic determined to impact a likelihood that a complication results from an arthroplasty procedure. The system obtains first candidate values for modifiable risk factors of the patient defining a mutable characteristic of the patient or the arthroplasty procedure. The first candidate values are determined to minimize the likelihood that the complication results from the arthroplasty procedure. The system obtains second candidate values for the modifiable risk factors of the patient determined to maximize the likelihood that the complication results from the arthroplasty procedure. The system determines a personalized risk interval that represents a range of modifiable risk for the patient with respect to the arthroplasty procedure including determining (i) a lower bound and (ii) an upper bound for the personalized risk interval; and providing an output indicative of the risk interval.

### CLAIM OF PRIORITY

This application claims priority under 35 U.S.C. § 119 (e) to U.S. Patent Application Ser. No. 63/453,366, filed on Mar. 20, 2023, the entire contents of which are hereby incorporated by reference.

### BACKGROUND

### 1. Technical Field

This specification relates to calculation of periprosthetic fracture risk associated with total hip arthroplasty, including techniques for determination of risk values associated with non-operative and operative risk factors, image-based feature extraction, and generation and presentation of risk tables.

### 2. Background Discussion

Periprosthetic femur fracture (PPFFx) remains one of the most common and challenging problems associated with total hip arthroplasty (THA). Indeed, recent data from the American Joint Replacement Registry (AJRR) indicates it is the second most frequent indication for early revision THA (behind infection), which is corroborated by institutional and international registry data.

### SUMMARY

This specification describes systems, methods, devices, and techniques for determining personalized risk assessments for complications of total hip arthroplasty, including PPFFx.

In a first aspect, a computer-implemented method performed by a computing system includes obtaining values for one or more non-modifiable risk factors of a patient. The non-modifiable risk factors each define an immutable patient characteristic determined to impact a likelihood that a complication results from an arthroplasty procedure planned for the patient. The system further can further obtain first candidate values for one or more modifiable risk factors of the patient, each modifiable risk factor defining a mutable characteristic of the patient or the arthroplasty procedure determined to impact a likelihood that the complication results from the arthroplasty procedure planned for the patient. The first candidate values are determined to minimize the likelihood that the complication results from the arthroplasty procedure. The system can further obtain second candidate values for the one or more modifiable risk factors of the patient, where the second candidate values are determined to maximize the likelihood that the complication results from the arthroplasty procedure. The system determines a personalized risk interval that represents a range of modifiable risk for the patient with respect to the arthroplasty procedure. Determining the personalized risk interval can include determining (i) a lower bound for the personalized risk interval based on the values for the one or more non-modifiable risk factors of the patient and the first candidate values for the one or more modifiable risk factors and (ii) an upper bound for the personalized risk interval based on the values for the one or more non-modifiable risk factors of the patient and the second candidate values for the one or more modifiable risk factors; and providing an output indicative of the risk interval.

These and other aspects can further include one or more of the following features.

In an example, determining the lower bound for the personalized risk interval can include processing, with a machine-learning model, the values for the one or more non-modifiable risk factors of the patient and the first candidate values for the one or more modifiable risk factors; and determining the upper bound for the personalized risk interval can include processing, with the machine-learning model, the values for the one or more non-modifiable risk factors of the patient and the second candidate values for the one or more modifiable risk factors. The machine-learning model can include at least one of a regression model, a nomogram, an artificial neural network, a transformer model, or an XGBoost model.

In an example, the arthroplasty procedure can be a total hip arthroplasty procedure, the complication can be PPFFx, and the one or more non-modifiable risk factors can include at least one of a sex of the patient, an age of the patient, an indication of whether the patient has been diagnosed with osteoporosis or uses osteoporosis medication, and an indication of surgery for the patient other than osteoarthritis. The sex of the patient can be associated with values that include male and female; the age of the patient can be associated with values that include a number of years or decades since the patient's birth; the indication of whether the patient has been diagnosed with osteoporosis or uses osteoporosis medication is associated with values that include a positive indication of osteoporosis diagnosis or use of osteoporosis medication or a negative indication of osteoporosis diagnosis or use of osteoporosis medication; and the indication of surgery for the patient other than osteoarthritis is associated with values that include osteoarthritis, fracture, osteonecrosis, and inflammatory arthritis.

In an example, the arthroplasty procedure is a total hip arthroplasty procedure, the complication is PPFFx, and the one or more modifiable risk factors can include at least one of femoral fixation method, femoral implant type, or surgical approach. The femoral fixation method is associated with values of cemented and non-cemented; the femoral implant type is associated with values of collared and collarless; and the surgical approach is associated with values of direct anterior, lateral, and posterior.

In an example, the method can include for each of the one or more modifiable risk factors: identifying a first value of the modifiable risk factor associated with a lowest risk of the complication from the arthroplasty procedure among all possible values for the modifiable risk factor; and selecting the first value for inclusion in the first candidate values. The computer-implemented method can include for each of the one or more modifiable risk factors: identifying a second value of the modifiable risk factor associated with a highest risk of the complication from the arthroplasty procedure among all possible values for the modifiable risk factor; and selecting the second value for inclusion in the second candidate values.

In an example, the patient is a human.

In an example, the method can include obtaining a set of user-specified values for the one or more modifiable risk factors of the patient; and determining a personalized, modifiable risk score for the patient based on the values for the one or more non-modifiable risk factors of the patient and the set of user-specified values for the one or more modifiable risk factors. Providing the output indicative of the risk interval can include displaying an indication of the risk interval, storing the indication of the risk interval, or transmitting the indication of the risk interval to a remote computing system. Providing the output indicative of the risk interval can include generating computer code can include instructions that, when executed, cause an indication of the risk interval to be presented in an interactive user interface on a screen of an electronic device.

Where the arthroplasty procedure is a total hip arthroplasty procedure, the complication is PPFFx, and the one or more non-modifiable risk factors can include image features extracted from one or more pre-operative images of a femoral or pelvic region of the patient. The method can include extracting the image features using a machine-learning model trained to predict whether or a likelihood that a patient exhibits PPFFx following the total hip arthroplasty procedure based on one or more pre-operative images of the femoral or pelvic region.

In another aspect, a computer-implemented method includes obtaining user-indicated values for one or more non-modifiable risk factors of a patient, each non-modifiable risk factor defining an immutable patient characteristic that is determined to impact a likelihood that a complication results from an arthroplasty procedure that is planned for the patient; obtaining user-indicated values for one or more modifiable risk factors of the patient, each modifiable risk factor defining a mutable characteristic of the patient or the arthroplasty procedure that is determined to impact the likelihood that the complication results from the arthroplasty procedure that is planned for the patient; determining a personalized, modifiable risk score for the patient with respect to the complication and the arthroplasty procedure based on the user-indicated values for the one or more non-modifiable risk factors of the patient and the user-indicated values for the one or more modifiable risk factors; and providing an output indicative of the personalized, modifiable risk score for the patient.

These and other aspects can further include one or more of the following features.

In an example, determining the personalized, modifiable risk score can include processing, with a machine-learning model, the user-indicated values for the one or more non-modifiable risk factors of the patient and the user-indicated values for the one or more modifiable risk factors.

The arthroplasty procedure can be a total hip arthroplasty procedure, and the complication can be PPFFx.

The one or more non-modifiable risk factors can include at least one of a sex of the patient, an age of the patient, an indication of whether the patient has been diagnosed with osteoporosis or uses osteoporosis medication, and an indication of surgery for the patient other than osteoarthritis.

Where the arthroplasty procedure is a total hip arthroplasty procedure, the complication can be PPFFx, and the one or more non-modifiable risk factors can include image features extracted from one or more pre-operative images of a femoral or pelvic region of the patient. The computer-implemented method can include extracting the image features using a machine-learning model trained to predict whether or a likelihood that a patient exhibits PPFFx following the total hip arthroplasty procedure based on one or more pre-operative images of the femoral or pelvic region.

In an aspect, a system comprises circuitry configured to perform any of the methods disclosed herein. The circuitry can include software, hardware, digital electronics, analog electronics, or a combination of these.

In an aspect, one or more non-transitory computer-readable media are encoded with instructions that, when executed by one or more processors, cause the one or more processors to perform the actions, methods, and processes disclosed herein.

Additional features and advantages will be apparent to one of ordinary skill in view of the specification, the figures, and claims.

### BRIEF DESCRIPTION OF DRAWINGS

FIG. 1 is a schematic diagram of an example system for presenting calculated PPFFx risk associated with total hip arthroplasty on a user device.

FIG. 2 is an image depicting an example user interface for the inputting information relating to non-modifiable medical information for calculating a PPFFx risk nomogram.

FIG. 3 is an image depicting an example user interface for viewing the PPFFx risk nomogram based on entered non-modifiable medical information.

FIG. 4 is a flow chart diagram depicting a process an example computer-implemented process for determining a personalized risk interval.

FIG. 5 is a flow chart diagram depicting a process for determining a personalized, modifiable risk score for a patient with respect to a complication and an arthroplasty procedure.

FIG. 6 is a table showing patient profiles with non-modifiable preoperative variables and modifiable intraoperative variables.

FIG. 7 is a table showing patient characteristics and primary THA performed for the patients of the study.

FIG. 8 is a table showing evaluated patient factors included in the final univariable and multivariable risk analysis models.

FIGS. 9A-9C are patient-specific PPFFx absolute risk nomograms for primary total hip arthroplasty at 90 days, 1 year, and 5 years, respectively.

FIGS. 10A-10B are nomogram for a hypothetical patient and various risk estimates in the setting of a primary THA for fracture (A) and osteoarthritis (B), respectively, including exemplary lines depicting how to use the nomogram.

FIG. 11 is a table showing PPFFx risk based on non-modifiable patient factors and modifiable operative decisions.

FIG. 12 is a schematic diagram of example computer systems for executing the methods and systems described herein.

### DETAILED DESCRIPTION

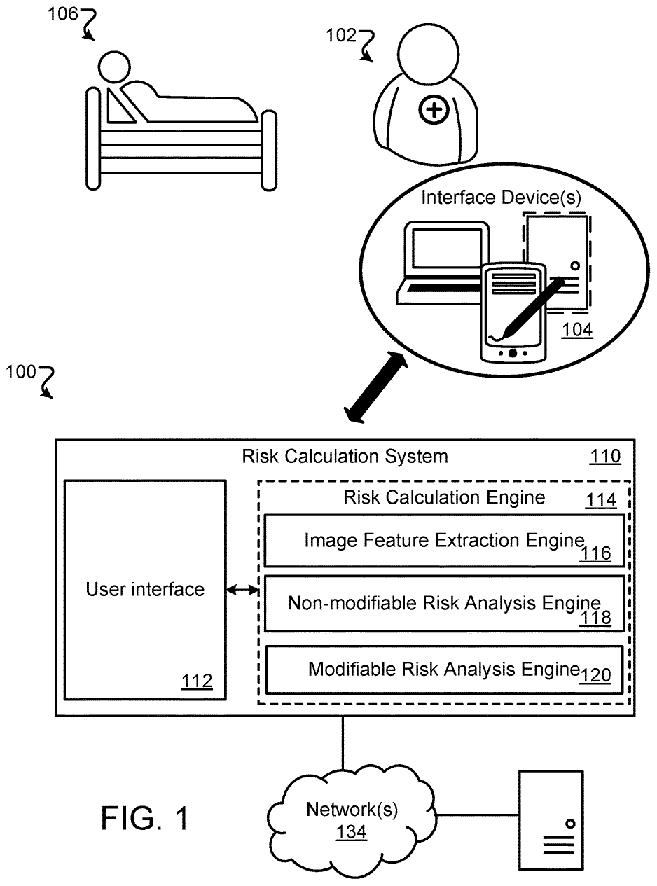

FIG. 1 is a block diagram of an example patient-specific PPFFx risk prediction system 100 that allows for dynamic risk modification based on non-operative data and operative decisions. In general, an operative decision is a discreet surgical method that can be pre-operatively selected which affects the risk a negative outcome of the associated medical procedure, such as a PPFFx following total hip arthroplasty. Within this specification, the term ‘operative’ refers to a class of ‘modifiable’ risk factors and the term ‘non-operative’ refers to a class of ‘non-modifiable’ risk factors. The example risk prediction system 100 can be used to calculate, e.g., determine, the risk of one or more complications resulting from a medical procedure which is planned for a patient. In the example system 100 described herein, the risk prediction system 100 is configured to calculate the risk of a PPFFx complication resulting from a THA procedure.

System 100 depicts a user 102 interacting with exemplary interface device 104. The user 102 in this example is a medical user, e.g., a medical professional, a surgeon, or medical assistant, although the techniques disclosed in this specification may be extended for use with other users as well. In the example of FIG. 1, the user 102 is screening a patient 106 for an arthroplasty procedure, e.g., a THA procedure, and utilizing the system 100 to determine a personalized risk interval, e.g., a nomogram, based on one or more non-modifiable and one or more modifiable, operable risk factors which can be made by the user 102 pre-operatively.

The user 102 interacts with the interface device 104 to input user-specified, non-modifiable risk factors into the system 100. The interface device 104 stores in non-transitory media the risk calculation system 110. The risk calculation system 110 includes a user interface 112 with which the user 102 interacts and inputs the non-modifiable risk factors into the model 110. The user interface 112 includes control elements for receiving the input from the user 102 such as radio buttons, text boxes, and/or other input fields into which the user 102 inputs the medical data for the patient 106.

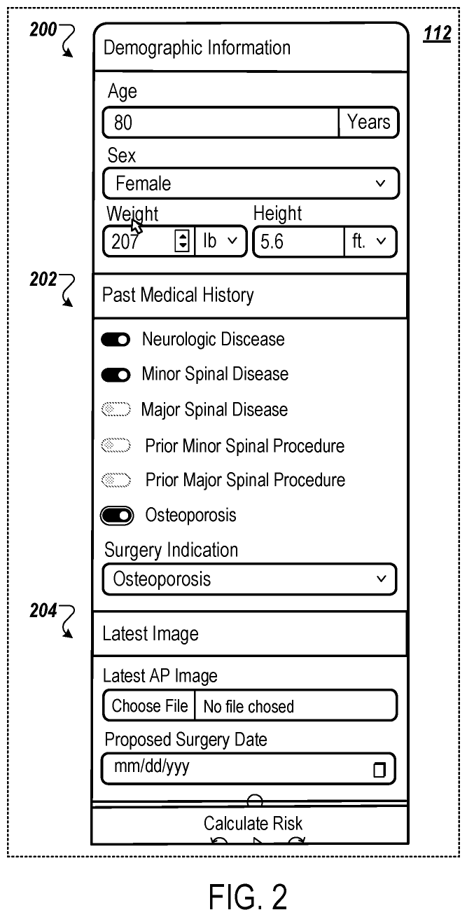

Referring to FIG. 2, exemplary user interface windows are shown which can be presented to the user 102 on the user interface 112. The exemplary user interface windows of FIG. 2 include a demographic information window 200, a past medical history window 202, and an image upload window 204. The demographic information window 200 includes input fields for some non-modifiable risk factors which can be user-specified values, or values received from a patient data management platform. The non-modifiable risk factors displayed in the demographic information window 200 are age, sex, weight, and height.

The past medical history window 202 includes binary input fields (e.g., binary sliders, radio buttons) for non-modifiable risk factors which have a binary value indicating the presence of absence of the associated non-modifiable risk factors. As with other non-modifiable risk factors, the binary risk factors can be user-specified values, or received from a patient data management platform. The binary non-modifiable risk factors displayed in the demographic information window 200 are neurologic disease, minor spinal disease, major spinal disease, prior minor spinal procedure, prior major spinal procedure, and indications of osteoporosis.

The values displayed in the demographic information window 304 and/or the medical history window 306 can be the values input into the user interface 112 in the demographic information window 200 or the medical history window 202, respectively.

The image upload window 204 includes a file selection field in which a user may select one or more image files containing image data for processing by the risk prediction system 100. The image file can be any medically-relevant image file, such as JPEG, PNG, or other medically-related image file which the image feature extraction engine 116 can process the image data and extract features from. For example, in a THA procedure, the user 102 may upload image files containing image data representing medical scans of a hip area of a patient detailing portions of the femoral head, femoral stem, or bones of the pelvic girdle.

In some examples, the user 102 uploads a data file containing image data from a medical scan to the image feature extraction engine 116. In some examples, the data files from the medical scan contain image data representing medical scans of the patient detailing parts of the body other than or including the hip area. Such data files can include an x-ray scan data file, a computed tomography (CT) scan data file, or a dual-energy x-ray absorptiometry (DXA) scan data file. The data files described herein can incorporate imaging interpretations of bone quality which can be used by the image feature extraction engine 116 or other modules, such as the non-modifiable risk analysis engine 118, or the modifiable risk analysis engine 120.

Referring again to FIG. 1, the user interface 112 receives the input indicative of non-operable medical data of the patient 106 and transmits the medical data to a risk calculation engine 114 which communicates with an image feature extraction engine 116, a non-modifiable risk calculation engine 118, and a modifiable risk calculation engine 120.

In another example, the system 100 receives non-modifiable patient data from a database, look up table, or other data storage system connected to a network 134 in communication with the system 100, such as a patient data management system which stores individualized, non-modifiable risk factors specific to the patient 106. The system 100 can receive the non-modifiable patient data from the network 134 alone or in combination with user-specified, non-modifiable risk factors input by the user 102.

The user interface 112 is communicatively connected to a risk calculation engine 114 which receives the modifiable and non-modifiable risk factors from the user interface 112. The risk calculation engine 114 includes an image feature extraction engine 116, a non-modifiable risk calculation engine 118, and a modifiable risk calculation engine 120. In some cases, the image feature extraction engine 116 is implemented in the non-modifiable risk calculation engine 118 since the image features can be considered non-modifiable risk factors. Each of the engines 116, 118, 120 can be implementations of suitable models trained to generate output based on the received input. In some examples, the models are machine-learning models.

The image feature extraction engine 116 is configured to receive pre-operative images, e.g., image data, from the user interface 112 and generate image features indicative of one or more risk factors of the procedure. The image feature extraction engine 116 receives image data and generates a set of features based on the received images which can be correlated with one or more surgical outcomes related to the modifiable options or non-modifiable risk factors. Examples of the image feature extraction engine 116 are survival machine-learning models, or multimodal survival machine-learning models which can process more than one type of input data.

One example of the image feature extraction engine 116 is XGBoost, an open-source implementation of the supervised learning, gradient-boosted trees algorithm which attempts to accurately predict a target variable by combining the estimates of a set of simpler, weaker models. The image feature extraction engine 116 can also be implemented with convolutional neural networks, transformers, or other machine-learning models. The image feature extraction engine 116 extracts the most informative imaging features from the image data and concatenates the image data with one or more clinical features from the received modifiable or non-modifiable patient data. The image feature extraction engine 116 is pre-trained using a collection of these features to output predicted individualized risk of PPFFx based on the features extracted from the clinical images.

The non-modifiable risk analysis engine 118 and the modifiable risk calculation engine 120 can be implementations of a multivariable regression model, e.g., a Cox proportional hazard models, configured to associate time to outcome events against a set of explanatory variables, e.g., the non-modifiable patient data. The non-modifiable risk calculation engine 118 is trained on patient data which included values for one or more of the non-modifiable risk factors. In some examples, the non-modifiable risk factors includes demographics, THA indication, bone quality, bone density, sex (e.g., but not limited to, male and female), age (e.g., a number of years or decades since the patient's birth), diagnosis of osteoporosis or use of osteoporosis medications (e.g., a positive or negative indication of osteoporosis diagnosis or use of osteoporosis medication), and indication for surgery other than osteoarthritis (e.g., osteoarthritis, fracture, osteonecrosis, and inflammatory arthritis), or comorbidities.

As an example, a hybrid network of EfficientNet-B4 and Swin-B transformer can be configured to classify patients based on n-year PPFFx outcomes (e.g., 1, 2, 5, or 10 year outcomes) from preoperative medical images, e.g., images of the anteroposterior (AP) pelvis radiographs, and clinical (demographics, comorbidities, and surgical) characteristics. The most informative imaging features, extracted by the mentioned model, can then be selected and concatenated with clinical features. A collection of these features can be used to train a multimodal survival XGBoost model to predict the individualized risk of PPFFx or other complication of interest.

The machine learning models used in the engines 116, 118, 120 can be trained with additional data. The data can include image files or data files received through the user interface or over the network 134. The image files or data files can include test results, modifiable risk factor data, non-modifiable risk factor data, image files, or data files containing image data. For example, the models can receive an x-ray scan data file, a CT scan data file, or a DXA scan data file and update the trained model with additional information.

The engine 120 receive the modifiable risk factor values from the user interface 112 and generates first candidate values which represents the minimum likelihood, and second candidate values which represents the maximum likelihood, that a complication such as a PPFFx results from the arthroplasty procedure.

The model 110 determines a personalized risk interval for the patient based on their values for the one or more non-modifiable risk factors with the first and second candidate values from the modifiable risk factor values. The interval is defined by an upper and a lower bound for the personalized risk interval using best and worst possible patient scenarios. The upper and lower bounds are based on the maximum and minimum complication likelihoods from the engines 118 and 120.

Based on the output from the engines 116, 118, 120, the model 110 generates an output indicative of the risk interval. The model 110 provides the output to the user interface 112 such that the risk calculation engine 114 presents the output for display to the user 102. In some examples, the model 110 provides the output to the interface device 104. Additionally or alternatively, the model 110 generates computer code including instructions that, when executed, cause an indication of the risk interval to be presented on the interface device 104.

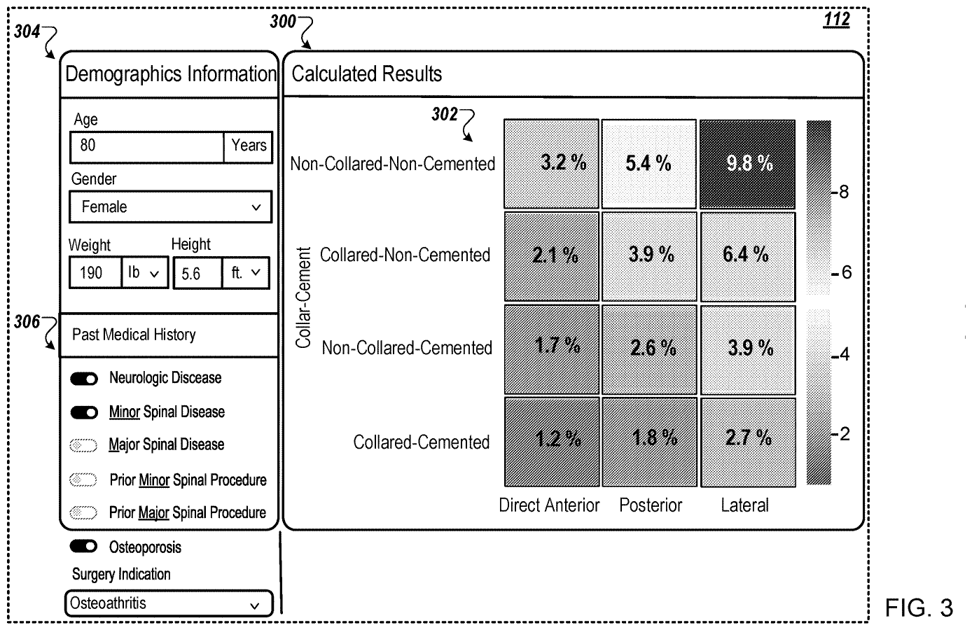

In one example, the output is a table of risk values comparing one or more of the modifiable risk factors against one or more of the remaining modifiable risk factors which can be presented on the user interface 112 of the interface device 104 to the user 102. Referring to FIG. 3, the output of the model 110 is indicated as displayed in the user interface 112 in a results window 300 titled ‘Calculator Results’ and presents a table 302 comparing values of two operable risk factors including femoral implant type (e.g., collared, or collarless), and method of femoral fixation (e.g., cemented, or uncemented) against surgical approach (e.g., direct anterior, lateral, or posterior). By presenting combinations of two risk factors along a first table axis against a single risk factor, the table presents an indication of which combination of three operable risk factors may lead to the least risk of negative outcomes, such as PPFFx. A heat-map legend is shown adjacent the table 302, indicating lowest risk values (e.g., <2) at the bottom and highest risk values (e.g., >8) at the top.

The table 302 indicates the combination of a non-collared femoral implant type with a non-cemented femoral fixation method and a lateral surgical approach has the highest risk factor based on the non-modifiable risk factors of 9.8% chance of a PPFFx within the selected time window. The table 302 indicates the combination of a collared femoral implant type with a cemented femoral fixation method and a direct anterior surgical approach has the lowest risk factor of 1.2% chance of a PPFFx within the selected time window.

A demographic information window 304 is shown adjacent the results window 300 which includes some values of the user-provided demographic information such as age, sex, weight, and height. Other values of the non-modifiable risk factors may also be displayed. A medical history window 306 is shown adjacent the results window 300 and demographic information window 304 which displays user-selected non-modifiable risk factors which are represented by binary values, e.g., presence or absence of the non-modifiable risk factor. In the example medical history window 306, binary values for neurologic disease, minor spinal disease, major spinal disease, prior minor spinal procedure, prior major spinal procedure, and indications of osteoporosis are shown.

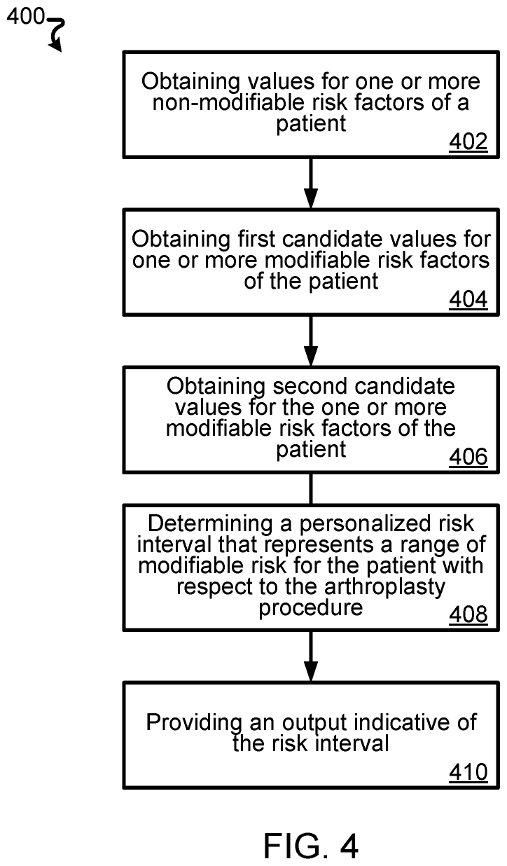

FIG. 4 is a flowchart of an example computer-implemented process 400 for determining a personalized risk interval that represents a range of modifiable risk for the patient with respect to the arthroplasty procedure and providing an output indicative of the risk interval.

The process 400 may be used, for example, by a medical user, e.g., user 102, for determining total risk from complications for patients, e.g., patient 106, undergoing an arthroplasty procedure, e.g., a total hip or other arthroplasty procedure. By inputting values for one or more non-modifiable or modifiable risk factors of the patient into the computer-implemented process 400, the process 400 determines a personalized risk interval that represents a range of modifiable risk for the patient with respect to the arthroplasty procedure.

A medical user obtains values for one or more non-modifiable risk factors of a patient (402). In general, the patient can be a human, though in other examples, the patient is an animal. In some implementations, each non-modifiable risk factor defines an immutable patient characteristic that is determined to impact a likelihood that a complication results from an arthroplasty procedure that is planned for the patient. Examples of the non-modifiable risk factors include any described herein. Some examples of the arthroplasty procedure include a total hip arthroplasty procedure, while an example of the complication can be a PPFFX.

In an example in which the arthroplasty procedure is a total hip arthroplasty procedure, and the complication is PPFFx, the non-modifiable risk factor includes image features extracted from one or more pre-operative images of a femoral or pelvic region of the patient. The image features can be extracted from the pre-operative image using a machine-learning model trained to predict whether, or a likelihood that, a patient exhibits PPFFx following the total hip arthroplasty procedure based on one or more pre-operative images of the femoral or pelvic region.

The medical user obtains first candidate values for one or more modifiable risk factors of the patient (404). Each modifiable risk factor defines a mutable characteristic of the patient or the arthroplasty procedure that is determined to impact the likelihood that the complication results from the arthroplasty procedure that is planned for the patient. The first candidate values are pre-operatively determined to minimize the likelihood that the complication results from the arthroplasty procedure.

The medical user obtains second candidate values for the one or more modifiable risk factors of the patient (406). The second candidate values are determined to maximize the likelihood that the complication results from the arthroplasty procedure. Examples of the first and/or second candidate values for the modifiable risk factors can include femoral fixation method (e.g., cemented and non-cemented), femoral implant type (e.g., collared and collarless), or surgical approach (e.g., direct anterior, lateral, and posterior).

The medical user enters the values for one or more non-modifiable risk factors, first candidate values, and second candidate values for the one or more modifiable risk factors of the patient into a patient-specific PPFFx risk prediction model that determines dynamic risk modification based on the non-modifiable and modifiable risk factors, such as system 100. In some examples of the process 400, the system identifies a first value of the modifiable risk factors which is associated with a lowest risk, and/or a second value associated with the highest risk, of the complication from the arthroplasty procedure among all possible values for the modifiable risk factor; and selects the first value, and/or the second value, for inclusion in the first and/or second candidate values.

The system determines a personalized risk interval that represents a range of modifiable risk for the patient with respect to the arthroplasty procedure (408). This includes determining (i) a lower bound for the personalized risk interval based on the values for the one or more non-modifiable risk factors of the patient and the first candidate values for the one or more modifiable risk factors and (ii) an upper bound for the personalized risk interval based on the values for the one or more non-modifiable risk factors of the patient and the second candidate values for the one or more modifiable risk factors.

The system determining the upper and/or lower bound for the personalized risk interval can include processing, with a machine-learning model, the values for the one or more non-modifiable risk factors of the patient and the first candidate values for the one or more modifiable risk factors. Some examples of the machine-learning model includes a regression model, a nomogram, an artificial neural network, a transformer model, or an XGBoost model.

The system provides an output indicative of the risk interval (410). This can include displaying an indication of the risk interval, such as to the user 102 on the interface device 104, storing the indication of the risk interval, or transmitting the indication of the risk interval to a remote computing system. The indication can be stored and/or transmitted locally, e.g., on the interface device 104, or to a remote computing system, e.g., to a networked computing system over the internet. In another example, this can include generating computer code including instructions that, when executed, cause an indication of the risk interval to be presented in an interactive user interface on a screen of an electronic device, e.g., interface device 104.

Optionally, the process 400 can include obtaining a set of user-specified values for the one or more modifiable risk factors of the patient; and determining a personalized, modifiable risk score for the patient based on the values for the one or more non-modifiable risk factors of the patient and the set of user-specified values for the one or more modifiable risk factors.

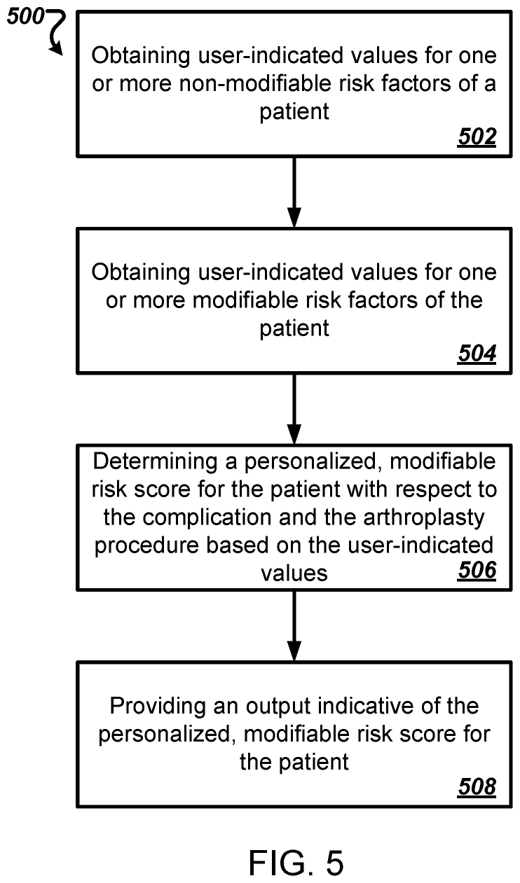

FIG. 5 is a flowchart of an example process 500 for determining a personalized, modifiable risk score for a patient, e.g., patient 106, with respect to a complication and an arthroplasty procedure based on user-indicated values for one or more non-modifiable and modifiable risk factors. In some examples, the arthroplasty procedure is a total hip arthroplasty procedure, and the complication is PPFFx.

The process 500 may be used, for example, by a medical user, e.g., user 102, for determining personalized, modifiable risk score from complications for the patient undergoing the arthroplasty procedure, e.g., a total hip or other arthroplasty procedure, using a rick calculation system, e.g., system 100.

A system obtains, from the user, user-indicated values for one or more non-modifiable risk factors of a patient (502). Each non-modifiable risk factor defines an immutable patient characteristic that is determined to impact a likelihood that a complication results from the arthroplasty procedure planned for the patient.

The system obtains, from the user, user-indicated values for one or more modifiable risk factors of the patient (504). Each modifiable risk factor defines a mutable characteristic of the patient or the arthroplasty procedure that is determined to impact the likelihood that the complication results from the arthroplasty procedure. In an example, the non-modifiable risk factors includes image features extracted from one or more pre-operative images of a femoral or pelvic region of the patient. Optionally, extracting the image features includes using a machine-learning model trained to predict whether, or a likelihood that, a patient exhibits PPFFx following the total hip arthroplasty procedure based on one or more pre-operative images of the femoral or pelvic region.

The system determines a personalized, modifiable risk score for the patient (506). The personalized, modifiable risk score is determined with respect to the complication and the arthroplasty procedure based on the user-indicated values for the one or more non-modifiable risk factors of the patient and the user-indicated values for the one or more modifiable risk factors. Determining the personalized, modifiable risk score can include processing, with a machine-learning model (e.g., any machine-learning model described herein), the user-indicated values for the one or more non-modifiable risk factors of the patient and the user-indicated values for the one or more modifiable risk factors.

The system provides an output indicative of the personalized, modifiable risk score for the patient (508). The system can provide the output in any manner described herein.

### Example Study

### Patients and Methods

Summary 16,696 primary non-oncologic THAs performed between 1998-2018 were evaluated. During a mean-6-year follow-up, 558 patients (3.3%) sustained a PPFFX. Patients were characterized by individual natural language processing assisted chart review on non-modifiable factors (demographics, THA indication, comorbidities), and modifiable operative decisions (femoral fixation [cemented/uncemented], surgical approach [direct anterior, lateral, posterior], implant type [collared/collarless]). Multivariable Cox regression models and nomograms were developed with PPFFx as a binary outcome at 90-days, 1-year, and 5-years postoperatively.

### Patients

Following Institutional Review Board approval, 16,696 primary non-oncologic THAs were evaluated from a single institutional total joint registry (TJR) from 1998-2018 with a mean 6 years of follow-up. Patients were characterized using a prospectively-collected total joint registry with augmentation to determine specific comorbidities and medication exposures of interest (osteoporosis, diabetes mellitus, end stage renal disease, malnutrition, liver disease, bariatric surgery, oral or intravenous steroids, chemotherapy, alcoholism, smoking) using diagnosis/procedure codes and natural language processing (NLP)-assisted chart review of the medical record with individual manual review of all diagnoses.

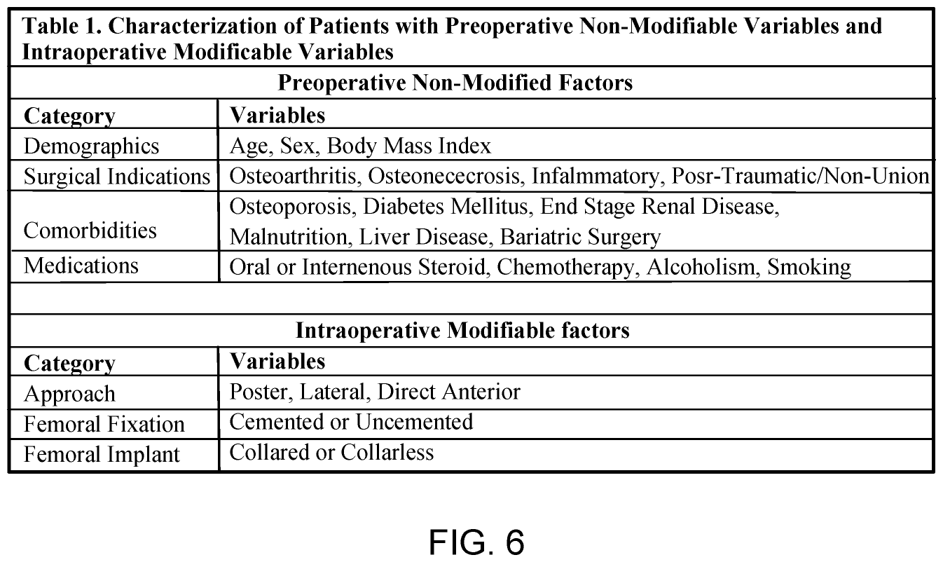

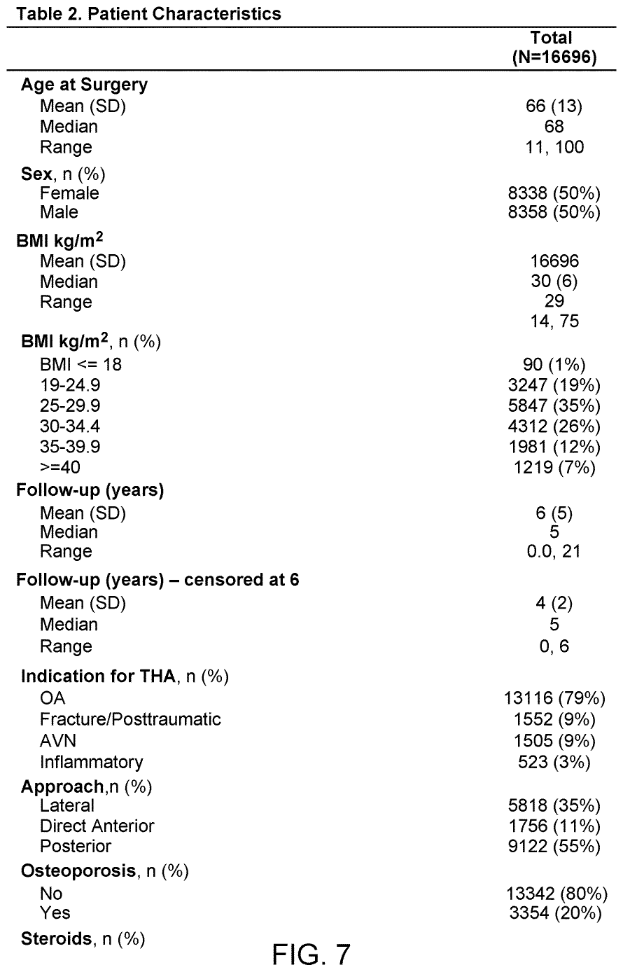

Analyzed surgical variables included: 1. femoral fixation (cemented/uncemented), 2. femoral implant type (collared/collarless), and 3. surgical approach (direct anterior, lateral, posterior) which enabled determination of patient profiles with non-modifiable pre-operative variables and modifiable intra-operative variables (FIG. 6). All cases were assumed to be at risk of PPFFx during or after THA and were followed until fracture, last follow-up, or death. All fractures were considered equivalent regardless of timing, location, or subsequent treatment. Univariable and multivariable Cox regression analysis determined hazard ratios (HRs) for variables associated with differential PPFFx risk. Since the study focused on PPFFx events within five years after surgery, follow-up was censored at a maximum of 6 years from THA. Variables that remained significant in multivariable analysis or improved the model fit based on the Akaike information criterion (AIC) were included in the final model.

A patient-specific PPFFx risk calculator was created with nomograms from multivariable modeling such that the individual risk for a patient with any combination of non-modifiable factors could be calculated and would determine differential risk based on modifiable operative decisions. These nomograms were built separately for 90-day, 1-year, and 5-year timepoints. Discrimination was assessed using the concordance statistic (c-statistic) for the Cox models. Calibration was assessed by comparing observed versus expected events in deciles of predicted risk using goodness-of-fit tests, which included standardized incidence ratios (SIR). All hazard ratios (HRs) reported below are statistically significant, with confidence intervals (CI) and p-values reported in the accompanying tables.

### Patient Characteristics and Operative Management

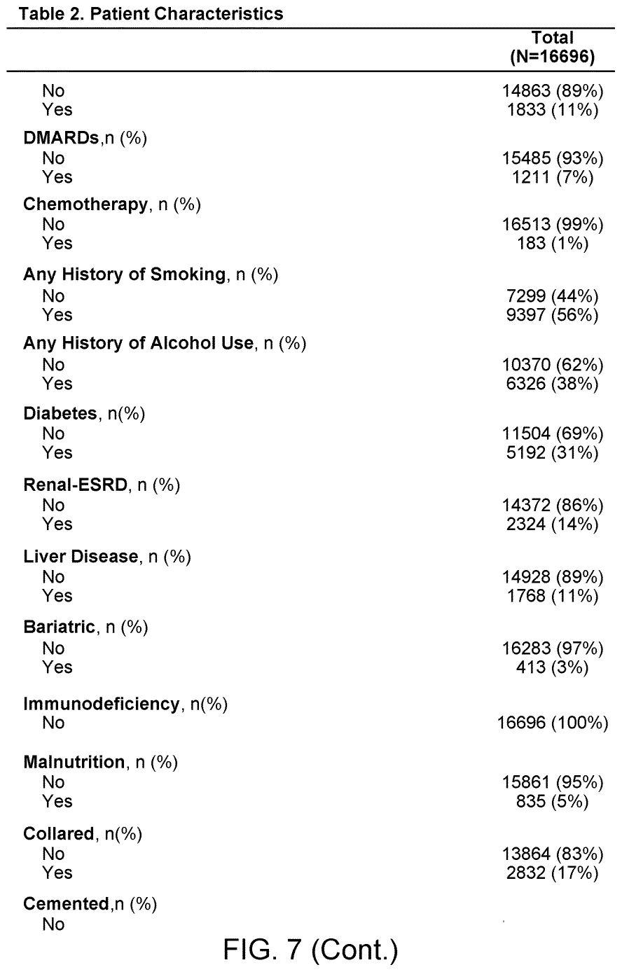

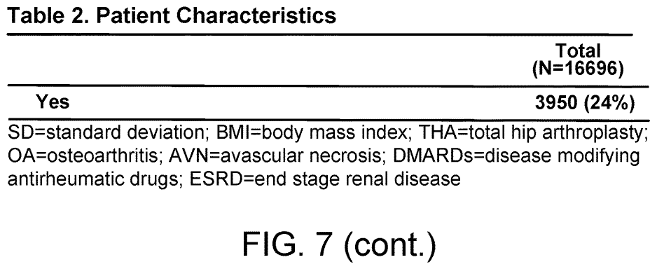

Mean patient age was 66 years (range, 12-100 years), mean body mass index (BMI) was 30 kg/m² (range, 14-75 kg/m²), and 50% patients were female (FIG. 7). Mean follow-up was 6 years (range, 2-21 years). History of evaluated comorbidities and medication exposures present at the time of THA was as follows: history of smoking (56%), alcoholism (38%), diabetes mellitus (31%), osteoporosis (20%), ESRD (14%), liver disease (11%), oral or intravenous steroids (11%), malnutrition (5%), bariatric surgery (3%), and chemotherapy (1%), (FIG. 7). Primary THA surgery was performed for osteoarthritis (79%), post-traumatic or fracture (9%), osteonecrosis (9%), and inflammatory arthritis (3%) (FIG. 7).

Primary THA was performed with a posterior approach in 55%, lateral approach in 35%, and direct anterior approach with 11%. Cemented femoral fixation was performed in 24% and collared femoral implants were used in 17% (FIG. 7).

### Results

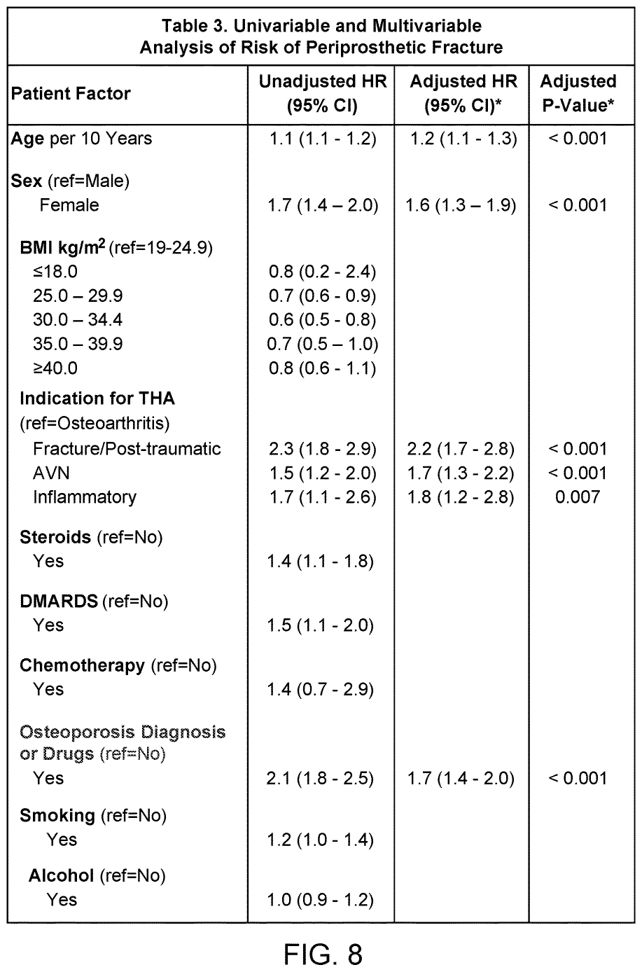

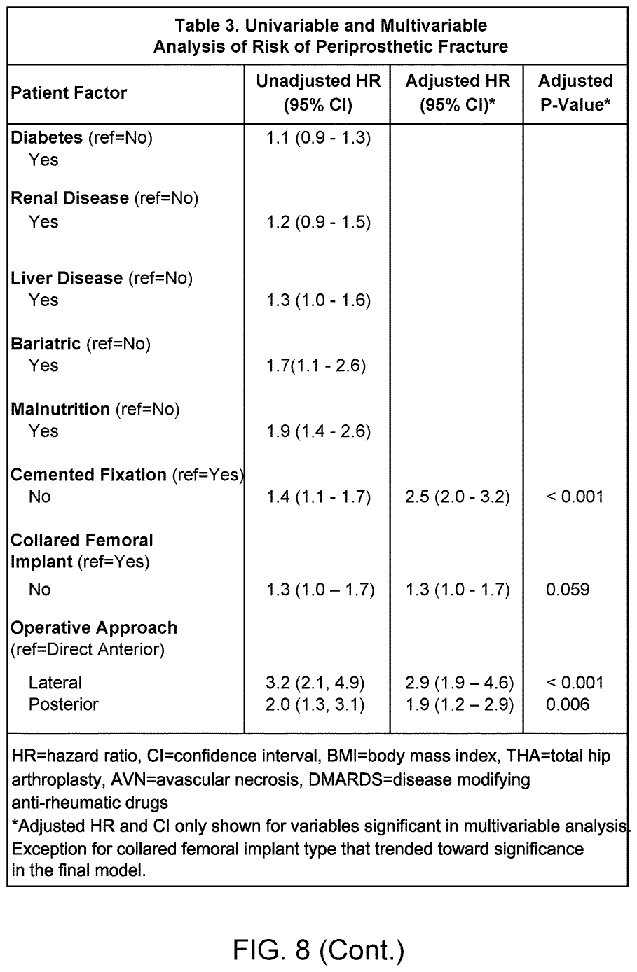

Among the 16,696 primary non-oncologic THAs, 558 patients sustained a PPFFx (5-year Kaplan-Meier survivorship rate of 3.7%). Among the 18 evaluated patient factors, 7 were included in the final multivariable model (FIG. 8). The 4 significant non-modifiable factors included: female sex (HR=1.6), older age (HR=1.2 per 10 years), diagnosis of osteoporosis or use of osteoporosis medications (HR=1.7), and indication for surgery other than osteoarthritis (HR=2.2 for fracture, HR=1.8 for inflammatory arthritis, HR=1.7 for osteonecrosis). All 3 analyzed modifiable surgical risk factors were included following multivariable analysis as follows: uncemented femoral fixation (HR=2.5), collarless femoral implants (HR=1.3), and surgical approach other than direct anterior (lateral HR=2.9, posterior HR=1.9).

The multivariable models demonstrated calibration SIR values of 0.98-0.99, consistent with “excellent” calibration. The model discrimination (concordance or C-statistic) values ranged from 0.68 to 0.69, consistent with “very good” discrimination.

### Nomogram Risk Calculator and Spectrum of Individual Patient Risk

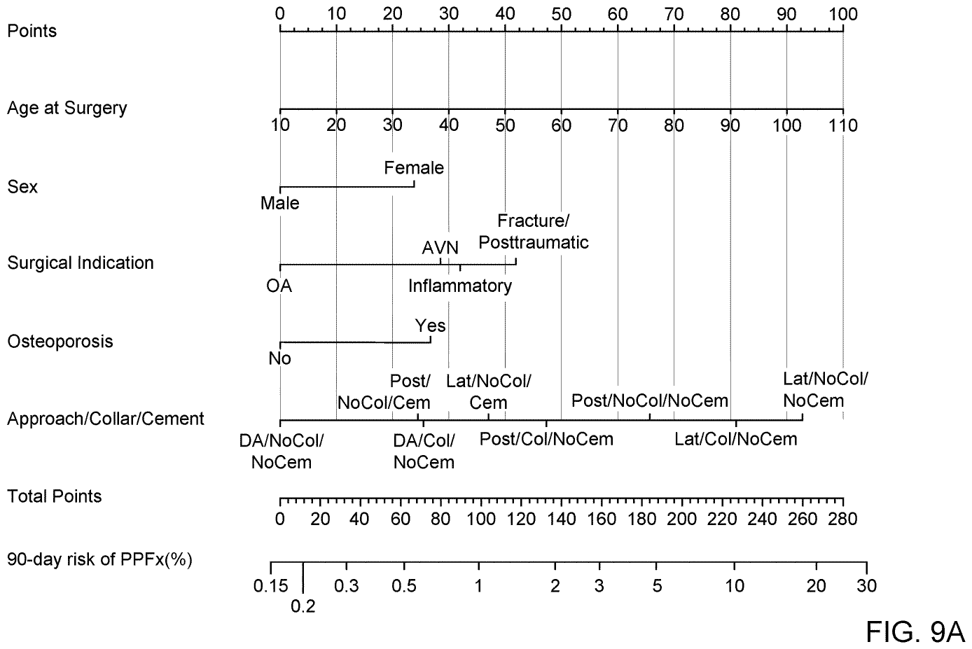

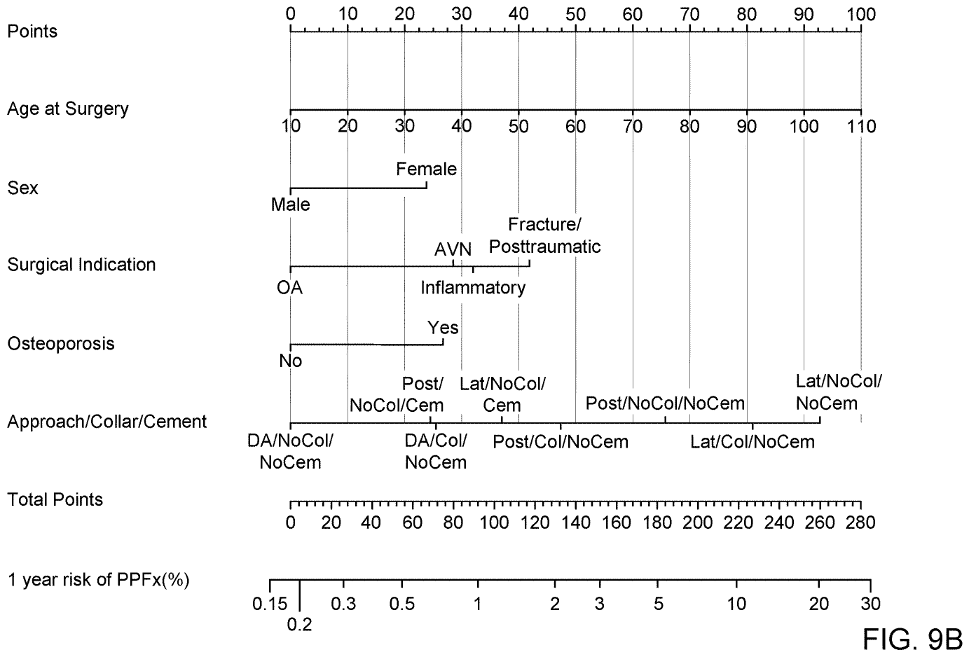

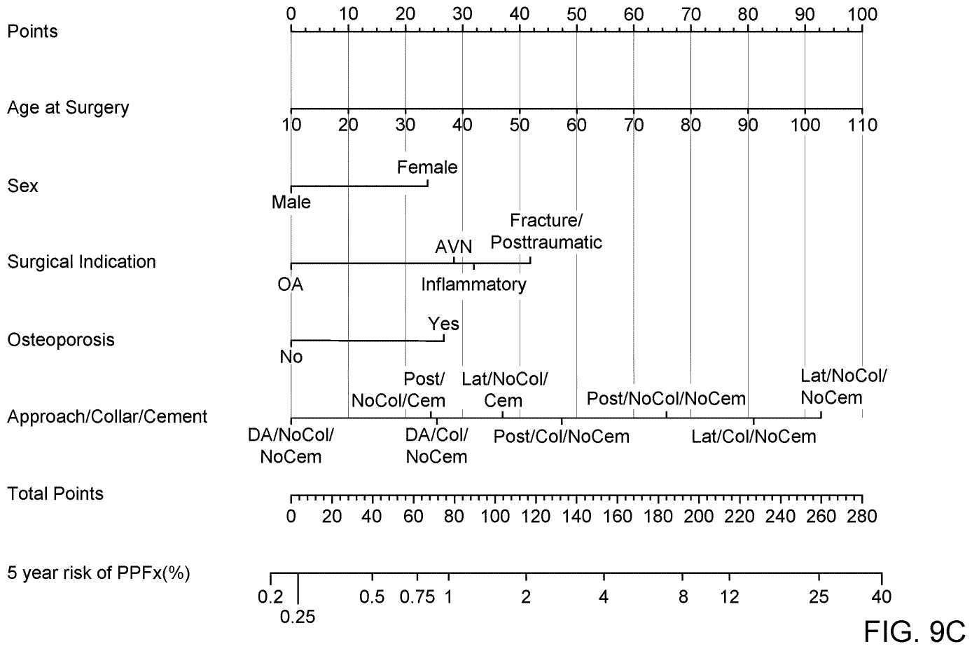

Nomograms of individual patient PPFFx risk were created from Cox proportional hazard models (FIGS. 9A-9C). Each patient factor is calibrated to be worth a certain number of points. Total points are calculated to obtain projected risk of PPFFx at 90 days, 1 year, and 5 years. The final data input line in the nomograms is approach, fixation method, and implant type. The combination of these 3 factors yields the greatest differential in possible point total, underscoring the power surgeons have to modify risk.

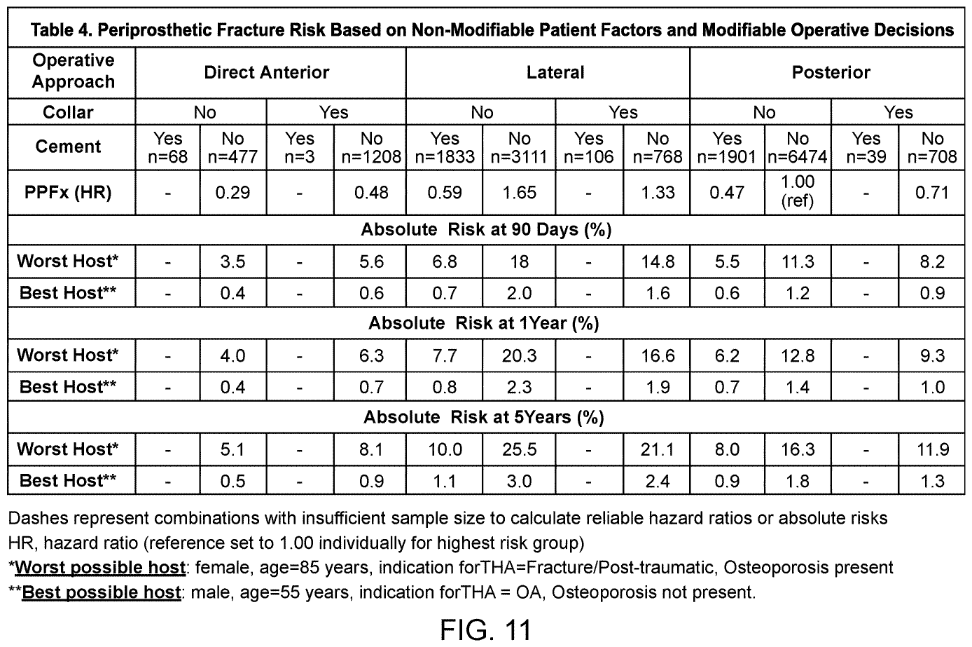

To understand the range of risk associated with non-modifiable patient factors, as well as the impact of modifiable operative decisions, a series of patient scenarios were created to define the upper and lower boundaries of the nomogram using best and worst possible patient scenarios (FIG. 11). Patient-specific PPFFx risk based on comorbid profile was wide-ranging from 0.4%-18% at 90-days, 0.4%-20% at 1-year, and 0.5%-2 mn5% at 5-years.

### Case Examples

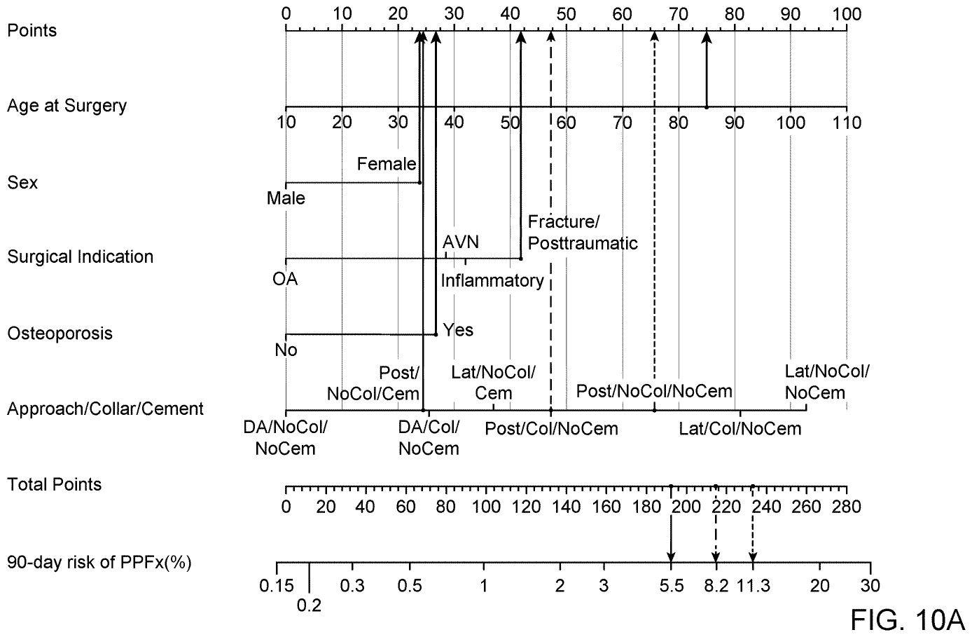

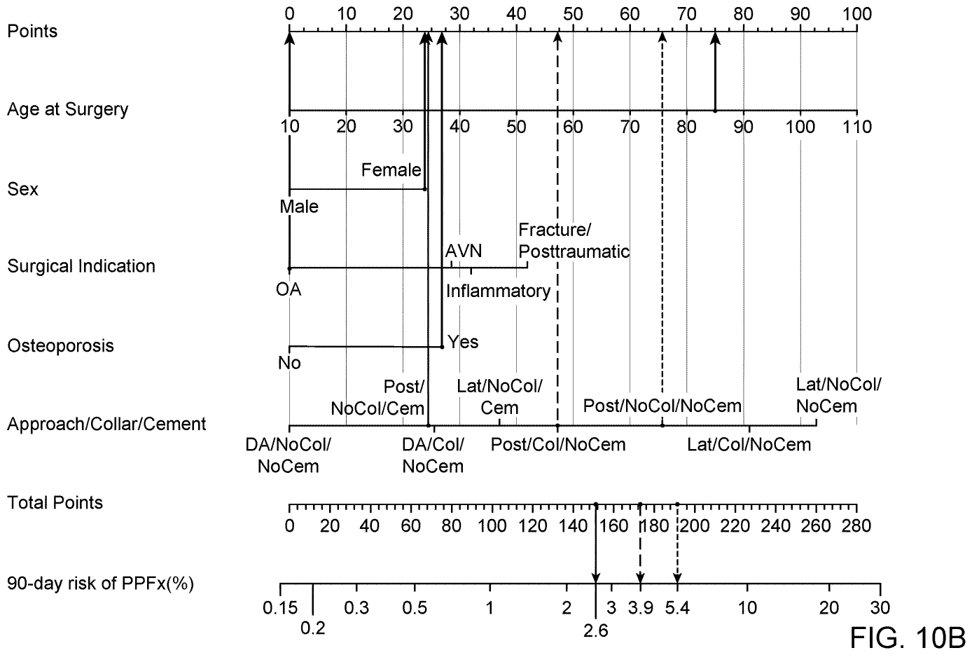

An 85-year-old female patient undergoes a THA for a fracture, and has a history of osteoporosis. The absolute risk of PPFFx at 90-days ranges from 3.5%-18% based on the patient comorbid profile, and final risk within that spectrum is based on operable choices within control of the surgeon. If the performing surgeon selects a posterior approach, an uncemented collarless stem yields the highest risk at 11.3%, which can be decreased to 8.2% with use of a collared implant or decreased to 5.5% with cemented fixation (FIG. 10A). Furthermore, risk for the patient using a lateral or direct anterior approach can be calculated in FIG. 11 or FIG. 10A.

Presume the same patient presents for a posterior approach THA, but the indication is for routine osteoarthritis as opposed to fracture. The absolute risk of PPFFx at 90 days ranges from 1.6%-8.7%. An uncemented collarless stem would yield the highest risk at 5.4%, which could be decreased to 3.9% with use of a collared implant or decreased to 2.6% with cemented fixation (FIG. 10B).

### Discussion

Periprosthetic femur fracture remains one of the most frequent complications and reasons for revision following THA. Individual patient risk is an amalgamation of non-modifiable characteristics and modifiable operative decisions. This study leveraged a large cohort of patients meticulously characterized across a range of PPFFx risk comorbidities to derive risk prediction nomograms that are patient-specific and responsive to operative decisions. Surgeons can use these prediction tools to forecast 90-day, 1-year, and 5-year probability of PPFFx and determine the impact of fixation technique, implant type, and operative approach for risk mitigation.

This cohort included thorough characterization of patients beyond traditional demographic and operative factors by including evaluation of several comorbidities and medication exposures which can be related to bone quality. These data were all individually and manually validated. The aforementioned diagnoses then supplemented the TJR that already tracks patient demographic, operative, and complication data with >98% capture. An ideal model should balance the competing demands of 1) providing optimal prediction (e.g., which can mean more variables), and 2) being parsimonious and user-friendly (e.g., which can mean only keeping certain influential variables). Among the broad array of assessed factors with peer-reviewed literature support, the final model was determined to be parsimonious. 4 of 15 non-modifiable factors were included in the final model following multivariable analysis and all are readily acertained in a routine workup: age, sex, history of osteoporosis, and indication for THA. The additional 3 modifiable risk factors in the model are controlled by the performing surgeon: fixation technique, implant type, and operative approach. It should be noted that implant type (e.g., collared vs collarless) trended toward significance in the final model. In various evaluated models, implant type was significant in some and trended toward significance in others and improved model fit based on the Akaike information criterion (AIC). Furthermore, implant type was included as a modifiable risk factor as mounting evidence exists on the protective nature of collars in PPFFx prevention.

Baseline risk of PPFFx was shown to be highly variable based on non-modifiable comorbidities and risk factors, demonstrated by comparing “worst case” and “best case” patients in FIG. 11 which underscores considering comorbid status to accurately classify patients. A message in the present application centers on the control for surgeons to influence outcomes based on operative decisions. Operative covariates were the most impactful nomogram variables by a substantial margin. An approach utilizing operative risk factors demonstrated an influence on PPFFx total risk. For surgeons that perform some combination of approaches in practice, the patient-specific PPFFx risk prediction system may afford an opportunity to more selectively employ one approach compared to another. However, for the many surgeons who default to a specific approach, the data provides highly actionable information. Fixation technique was the most important factor in the risk analysis model with implant type also being influential. For example, a predominantly posterior approach surgeon can see in FIG. 11 that using a collared implant can decrease risk by approximately 30% and cementing the femoral component can decrease risk by >50%.

Absolute risk is considered in addition to relative risk. The overall impact of a mitigation strategy is different for a patient with a baseline 90-day PPFFx risk of 1% versus 10%. In this example, undertaking an operative decision that reduces relative risk by 50% changes the absolute risk from 1% to 0.5% vs. 10% to 5% in hypothetical patients which underscores the actionable nature of this patient-specific PPFFx risk calculator, especially as it pertains to selective cementing of femoral components.

Realistically, cementing the femoral component is likely to remain a minority practice in the United States as currently <6% of elective THA and <17% of THA for acute fracture are cemented. However, the calculator described herein indicates comparative absolute risk may facilitate appropriate selective use of cementing to best serve high risk patients in accordance with evidence applied as individualized medicine.

The proportion of patients undergoing THA with relevant risk factors is increasing. PPFFx is a complication with a high 1-year mortality and demanding of scarce healthcare resources. It is currently the second most common reason for early revision in the AJRR and data clearly indicates the readily-available, facile, and cost-effective strategies to address this issue are underutilized. The National Health Service of the United Kingdom has recently undertaken an initiative known as Get It Right the First Time (GIRFT). Driving this program is the idea that performing the right surgery, on the right patient, at the right time, and in the right place avoids many otherwise preventable complications. GIRFT has been successful in the first few years in decreasing revision and re-revision rates, demonstrating a real-world proof-of-concept that employing patient-specific evidence-based decisions can improve quality and cost of THA care.

This study can be interpreted in light of potential limitations. First, these results represent the experience of single center. In particular, certain combinations of approach, fixation method, and implant type were too rare for inclusion, and may be common at other centers. Comparative analysis from data at other institutions and external validation will be helpful to create a more generalizable model. Secondly, PPFFx is a relatively rare event with multifactorial etiology. That combination, for any clinic prediction problem, makes model discrimination and calibration difficult. Our model had “very good” discrimination, despite the aforementioned challenges inherent to modeling problems like PPFFx and achieved “excellent” calibration, which is a testament to model fine-tuning. Thirdly, all fractures in this series were treated as equivalent regardless of timing, pattern, or subsequent treatment. Subclassifying on these variables would have been prohibitive to meaningful risk modeling.

This study may be the first study yielding a patient-specific PPFFx risk calculator. Modeling accounted for myriad potentially important comorbidities, yet the final model is quite parsimonious and actionable with factors that can be ascertained routinely. The resultant nomograms are responsive to fixation method, implant type, and operative approach decisions, and thus can be used as a screening tool to identify and individualize recommendations and treatment for THA patients. This is especially important given the wide range of individual patient risk identified in this study, and the degree of risk mitigation portended by various operative strategies. Nomograms from this work will serve as the underlying foundation for a digital clinical tool to calculate patient risk in a streamlined fashion.

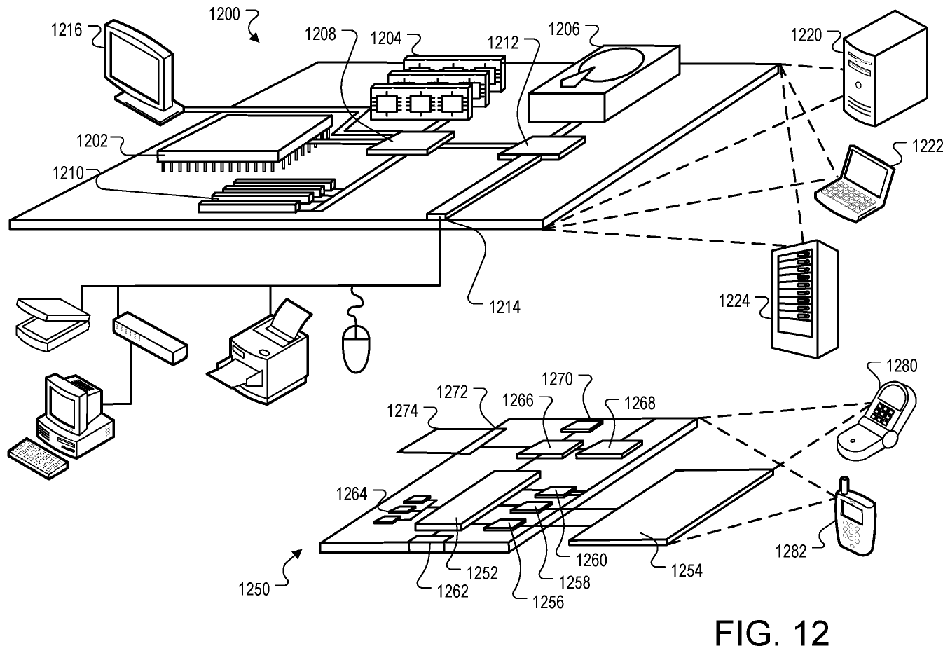

FIG. 12 shows an example computer system 1200 on which the patient-specific PPFFx risk prediction system 100 can be hosted that includes a processor 1200, a memory 1220, a storage device 1230 and an input/output device 1240. Each of the components 1210, 1220, 1230 and 1240 can be interconnected, for example, by a system bus 1250. The processor 1210 is capable of processing instructions for execution within the system 1200. In some implementations, the processor 1210 is a single-threaded processor, a multi-threaded processor, or another type of processor. The processor 1210 is capable of processing instructions stored in the memory 1220 or on the storage device 1230. The memory 1220 and the storage device 1230 can store information within the system 1200.

The input/output device 1240 provides input/output operations for the system 1200. In some implementations, the input/output device 1240 can include one or more of a network interface device, e.g., an Ethernet card, a serial communication device, e.g., an RS-232 port, and/or a wireless interface device, e.g., an 802.11 card, a 3G wireless modem, a 4G wireless modem, a 14G wireless modem, etc. In some implementations, the input/output device can include driver devices configured to receive input data and send output data to other input/output devices, e.g., keyboard, printer and display devices 1260. In some implementations, mobile computing devices, mobile communication devices, and other devices can be used.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (12AN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

12hile this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A computer-implemented method, comprising:
obtaining values for one or more non-modifiable risk factors of a patient, each non-modifiable risk factor defining an immutable patient characteristic that is determined to impact a likelihood that a complication results from an arthroplasty procedure that is planned for the patient;
obtaining first candidate values for one or more modifiable risk factors of the patient, each modifiable risk factor defining a mutable characteristic of the patient or the arthroplasty procedure that is determined to impact the likelihood that the complication results from the arthroplasty procedure that is planned for the patient, wherein the first candidate values are determined to minimize the likelihood that the complication results from the arthroplasty procedure;
obtaining second candidate values for the one or more modifiable risk factors of the patient, wherein the second candidate values are determined to maximize the likelihood that the complication results from the arthroplasty procedure;
determining a personalized risk interval that represents a range of modifiable risk for the patient with respect to the arthroplasty procedure, including determining (i) a lower bound for the personalized risk interval based on the values for the one or more non-modifiable risk factors of the patient and the first candidate values for the one or more modifiable risk factors and (ii) an upper bound for the personalized risk interval based on the values for the one or more non-modifiable risk factors of the patient and the second candidate values for the one or more modifiable risk factors; and
providing an output indicative of the risk interval.

2. The computer-implemented method of claim 1, wherein:
determining the lower bound for the personalized risk interval comprises processing, with a machine-learning model, the values for the one or more non-modifiable risk factors of the patient and the first candidate values for the one or more modifiable risk factors; and
determining the upper bound for the personalized risk interval comprises processing, with the machine-learning model, the values for the one or more non-modifiable risk factors of the patient and the second candidate values for the one or more modifiable risk factors.

3. The computer-implemented method of claim 2, wherein the machine-learning model comprises at least one of a regression model, an artificial neural network, a transformer model, or an XGBoost model.

4. The computer-implemented method of claim 1, wherein the arthroplasty procedure is a total hip arthroplasty procedure, the complication is periprosthetic femur fracture (“PPFFx”), and the one or more non-modifiable risk factors comprise at least one of a sex of the patient, an age of the patient, an indication of whether the patient has been diagnosed with osteoporosis or uses osteoporosis medication, and an indication of surgery for the patient other than osteoarthritis.

5. The computer-implemented method of claim 4, wherein:
the sex of the patient is associated with values that include male and female;
the age of the patient is associated with values that include a number of years or decades since the patient's birth;
the indication of whether the patient has been diagnosed with osteoporosis or uses osteoporosis medication is associated with values that include a positive indication of osteoporosis diagnosis or use of osteoporosis medication or a negative indication of osteoporosis diagnosis or use of osteoporosis medication; and
the indication of surgery for the patient other than osteoarthritis is associated with values that include osteoarthritis, fracture, osteonecrosis, and inflammatory arthritis.

6. The computer-implemented method of claim 1, wherein the arthroplasty procedure is a total hip arthroplasty procedure, the complication is periprosthetic femur fracture (“PPFFx”), and the one or more modifiable risk factors comprise at least one of femoral fixation method, femoral implant type, or surgical approach.

7. The computer-implemented method of claim 6, wherein:
the femoral fixation method is associated with values of cemented and non-cemented;
the femoral implant type is associated with values of collared and collarless; and
the surgical approach is associated with values of direct anterior, lateral, and posterior.

8. The computer-implemented method of claim 1, comprising for each of the one or more modifiable risk factors:
identifying a first value of the modifiable risk factor associated with a lowest risk of the complication from the arthroplasty procedure among all possible values for the modifiable risk factor; and
selecting the first value for inclusion in the first candidate values.

9. The computer-implemented method of claim 8, comprising for each of the one or more modifiable risk factors:
identifying a second value of the modifiable risk factor associated with a highest risk of the complication from the arthroplasty procedure among all possible values for the modifiable risk factor; and
selecting the second value for inclusion in the second candidate values.

10. The computer-implemented method of claim 1, wherein the patient is a human.

11. The computer-implemented method of claim 1, comprising:
obtaining a set of user-specified values for the one or more modifiable risk factors of the patient; and
determining a personalized, modifiable risk score for the patient based on the values for the one or more non-modifiable risk factors of the patient and the set of user-specified values for the one or more modifiable risk factors.

12. The computer-implemented method of claim 1, wherein providing the output indicative of the risk interval comprises displaying an indication of the risk interval, storing the indication of the risk interval, or transmitting the indication of the risk interval to a remote computing system.

13. The computer-implemented method of claim 1, wherein providing the output indicative of the risk interval comprises generating computer code comprising instructions that, when executed, cause an indication of the risk interval to be presented in an interactive user interface on a screen of an electronic device.

14. The computer-implemented method of claim 1, wherein the arthroplasty procedure is a total hip arthroplasty procedure, the complication is periprosthetic femur fracture (“PPFFx”), and the one or more non-modifiable risk factors comprise image features extracted from one or more pre-operative images of a femoral or pelvic region of the patient.

15. The computer-implemented method of claim 14, comprising extracting the image features using a machine-learning model trained to predict whether or a likelihood that a patient exhibits PPFFx following the total hip arthroplasty procedure based on one or more pre-operative images of the femoral or pelvic region.

16. One or more non-transitory computer-readable media encoded with instructions that, when executed by one or more processors, cause the one or more processors to perform the method of claim 1.

17. A system, comprising:
one or more processors; and
one or more computer-readable media encoded with instructions that, when executed by the one or more processors, cause the one or more processors to perform the method of claim 1.

18. A computer-implemented method, comprising:
obtaining user-indicated values for one or more non-modifiable risk factors of a patient, each non-modifiable risk factor defining an immutable patient characteristic that is determined to impact a likelihood that a complication results from an arthroplasty procedure that is planned for the patient;
obtaining user-indicated values for one or more modifiable risk factors of the patient, each modifiable risk factor defining a mutable characteristic of the patient or the arthroplasty procedure that is determined to impact the likelihood that the complication results from the arthroplasty procedure that is planned for the patient;
determining a personalized, modifiable risk score for the patient with respect to the complication and the arthroplasty procedure based on the user-indicated values for the one or more non-modifiable risk factors of the patient and the user-indicated values for the one or more modifiable risk factors; and
providing an output indicative of the personalized, modifiable risk score for the patient.

19-22. (canceled)

23. One or more non-transitory computer-readable media encoded with instructions that, when executed by one or more processors, cause the one or more processors to perform the method of claim 18.

24. A system, comprising:
one or more processors; and
one or more computer-readable media encoded with instructions that, when executed by the one or more processors, cause the one or more processors to perform the method of claim 18.
